(19) United States
(12) Patent Application Publication
(10) Pub. No.: US 2023/0365623 A1
(43) Pub. Date: Nov. 16, 2023

(54) DERIVATISATION OF PEPTIDES AND PROTEINS

(71) Applicant: FLOREY INSTITUTE OF NEUROSCIENCE AND MENTAL HEALTH, Parkville (AU)

(72) Inventors: Mohammed Akhter Hossain, Brunswick West (AU); John Wade, Canterbury (AU)

(21) Appl. No.: 18/024,631

(22) PCT Filed: Sep. 4, 2020
(86) PCT No.: PCT/AU2020/050934

§ 371 Date: Mar. 3, 2023

(51) Int. Cl. C07K 1/107

(52) U.S. Cl. CPC C07K 1/1077

(57) ABSTRACT

The present disclosure relates to methods for preparing derivatives of peptides and proteins, including preparing sialylglycosylated peptides and proteins, and to the resulting derivatives of peptides and proteins and their uses. The disclosure further relates to methods of reducing fibrillation of a peptide or protein by preparing derivatives of said peptides or proteins. The disclosure further relates to stable analogues of peptides and proteins, in particular stable insulin and glucagon analogues, which find use in methods of preparing derivatives of said peptides and proteins.

### TECHNICAL FIELD

The present disclosure broadly relates to methods of derivatising peptides and proteins, and to methods of inhibiting fibrillation of peptides and proteins by glycosylation. Exemplary embodiments relate to glycosylated insulin and glucagon analogues and derivatives that are not subject to fibrillation, and to methods of preparation of such analogues and derivatives.

### BACKGROUND OF THE DISCLOSURE

In the past 20 years, significant advances have been made in the development of insulin analogues for the management of diabetes mellitus. However, insulin analogues form oligomers and amyloid-type fibrils both in vitro and in vivo which reduces the shelf life of pharmaceutical preparations. This is especially problematic for patients that use external insulin pumps wherein fibrils can block the insulin delivery catheter, potentially leading to underdosing. The scale of the problem with fibrillation of insulin is enormous in terms of wastage of ‘expired’ drug. For example, in the US, savings of more than US$1 billion per year could be realised if insulin expiry dates were increased from just two days to six days for insulin pump users alone.

Fibrillation is also an obstacle present in the use of the peptide hormone glucagon. Glucagon is used to treat hypoglycemia in patients with diabetes. Like human insulin, glucagon is also known to form amyloid-like fibril structures at the acidic pH required for solubility and is therefore typically formulated as a lyophilised powder. Glucagon also forms fibrils at the physiological pH that has detrimental effects on its efficacy as a drug.

Fibrils formed by peptides and proteins are typically insoluble and potentially toxic. Fibrillation of particular peptide and protein drugs is especially likely at high concentration and/or high temperature. It is particularly desirable to prevent fibrillation at high temperature for use in poorer tropical regions, where access to reliable refrigeration may be limited.

The development of non-fibrillogenic analogues of insulin, glucagon, and other peptide and protein drugs is thus of high importance. It is desirable to provide non-fibrillogenic peptide and protein drugs, in particular insulin and glucagon analogues, especially which may be produced by an efficient and reproducible method.

### SUMMARY OF THE DISCLOSURE

The present disclosure is predicated on the inventors' unexpected discovery that peptides and proteins may be efficiently derivatised, especially glycosylated, with high specificity of the site of glycosylation, by means of introduction of a cysteine residue to the peptide or protein, followed by glycosylation via an S_{N}2 reaction between the thiol group of the cysteine and a glycosyl moiety having a leaving group. Further, the present disclosure is predicated on the discovery that an insulin analogue comprising inter-and intra-chain disulfide bridges and a single unprotected cysteine is stable, and a useful precursor for a glycosylation reaction or other derivatisation. Similarly, a stable glucagon analogue comprising a substituted amino acid having an unprotected nucleophilic group, in particular a cysteine residue, has been prepared and is a useful precursor for a glycosylation reaction or other derivatisation.

According to a first aspect, the present disclosure provides a method for glycosylating a peptide or protein, comprising the steps of: a) providing a peptide or protein comprising an amino acid residue comprising an unprotected nucleophilic group; and b) reacting the nucleophilic group with a glycosyl moiety substituted with a leaving group to provide a glycosylated peptide or protein.

In some embodiments, the peptide or protein is selected from the group consisting of insulin and glucagon, and derivatives, variants and fragments thereof.

In some embodiments, the method does not involve use of an enzyme.

In some embodiments, the glycosyl moiety is a sialylglycosyl moiety. In some embodiments, the glycosyl moiety substituted with a leaving group is a bromo-acetamidyl oligosaccharide. In some embodiments, the glycosyl moiety does not comprise glucose.

In some embodiments, the amino acid residue comprising an unprotected nucleophilic group is a cysteine or a lysine reside, in particular a cysteine residue. In some embodiments, the peptide or protein for use in step a) is modified from the native peptide or protein by introduction of the amino acid residue comprising an unprotected nucleophilic group as an additional residue or an amino acid substitution.

In embodiments in which the peptide to be glycosylated is insulin or an analogue thereof the amino acid residue comprising an unprotected nucleophilic group, optionally a cysteine residue, may be incorporated at the N-terminal of the B chain. In some embodiments, the peptide to be glycosylated is glucagon or an analogue thereof and the amino acid residue comprising an unprotected nucleophilic group is incorporated in substitution of the tryptophan residue at position 25 of native glucagon.

In some embodiments, the modified version of the peptide or protein is prepared by solid-phase peptide synthesis.

In some embodiments, step a) comprises the following steps:
a1) protecting the nucleophilic group of the amino acid residue with a protecting group;
a2) preparing a modified version of the peptide or protein comprising the amino acid residue, wherein the nucleophilic group of the amino acid residue is protected; and then
a3) deprotecting the nucleophilic group.

In some embodiments, at least 95% of the produced glycosylated peptide or protein is glycosylated in a single position on the peptide or protein.

According to a second aspect, the present disclosure provides a glycosylated peptide or protein obtained by a method of the first aspect.

According to a third aspect, the present disclosure provides a method of inhibiting fibrillation of a peptide or protein, comprising glycosylating the peptide or protein by the method according to the first aspect.

According to a fourth aspect, the present disclosure provides an insulin analogue comprising inter-and intra-chain disulfide bridges and at least one cysteine residue. In some embodiments, the cysteine residue is located at the N-terminal of the insulin B-chain. In some embodiments, the thiol group of the cysteine residue is protected by a protecting group. In some alternative embodiments, the cysteine residue is an unprotected cysteine residue.

According to a fifth aspect, the present disclosure provides use of an insulin analogue according to the fourth aspect in a method of preparing an insulin derivative. In some embodiments, the insulin derivative is a glycoinsulin.

According to a sixth aspect, the present disclosure provides a method of preparing a glycoinsulin comprising reacting the insulin analogue of the fourth aspect with a glycosyl moiety substituted with a leaving group.

According to a seventh aspect, the present disclosure provides a glucagon analogue, comprising a cysteine residue in substitution of a native amino acid residue. In some embodiments, the cysteine residue is substituted in place of the tryptophan residue at position 25 in native glucagon. In some embodiments, the thiol group of the cysteine residue is protected by a protecting group. In some alternative embodiments, the cysteine residue is an unprotected cysteine residue.

According to an eighth aspect, the present disclosure provides use of a glucagon analogue according to the seventh aspect in a method of preparing a glucagon derivative. In some embodiments, the glucagon derivative is a glycoglucagon.

According to a ninth aspect, the present disclosure provides a method of preparing a glycoglucagon comprising reacting the glucagon analogue of the seventh aspect with a glycosyl moiety substituted with a leaving group.

According to a tenth aspect, the present disclosure provides a method of treating diabetes and/or hyperglycaemia, comprising administering a therapeutically effective dose of a glycosylated insulin obtained by a method of the first aspect to a subject in need thereof.

According to an eleventh aspect, the present disclosure provides a method of lowering blood sugar levels, comprising administering a therapeutically effective dose of a glycosylated insulin obtained by a method of the first aspect to a subject in need thereof.

According to a twelfth aspect, the present disclosure provides use of a glycosylated insulin obtained by a method of the first aspect in the manufacture of a medicament for the treatment of diabetes and/or hyperglycaemia.

According to a thirteenth aspect, the present disclosure provides a method of treating hypoglycaemia, comprising administering a therapeutically effective dose of a glycosylated glucagon obtained by a method according to the first aspect to a subject in need thereof.

According to a fourteenth aspect, the present disclosure provides a method of raising blood sugar levels, comprising administering a therapeutically effective dose of a glycosylated glucagon obtained by a method of the first aspect to a subject in need thereof.

According to a fifteenth aspect, the present disclosure provides use of a glycosylated glucagon obtained by a method according to the first aspect in the manufacture of a medicament for the treatment of hypoglycaemia.

According to a sixteenth aspect, the present disclosure provides a sialylglycosylated insulin, or an analogue, variant or derivative thereof, wherein the sialylglycosylated insulin has a reduced tendency to fibrillate relative to native insulin.

According to a seventeenth aspect, the present disclosure provides a sialylglycosylated glucagon, or an analogue, variant or derivative thereof, wherein the sialylglycosylated glucagon has a reduced tendency to fibrillate relative to native glucagon.

According to an eighteenth aspect, the present disclosure provides a method of preparing a glycoinsulin comprising: a) providing a modified insulin analogue comprising an additional cysteine residue located at the N terminal of the B chain of the insulin; and b) reacting the thiol group of the cysteine residue with a glycosyl moiety substituted with a leaving group to provide a glycoinsulin. The glycosyl moiety may optionally be a sialylglycosyl moiety. Step a) may optionally comprise a1) protecting the thiol group of the cysteine residue with a protecting group; a2) preparing the modified insulin analogue comprising the additional cysteine residue, wherein the thiol group of the cysteine residue is protected; and then a3) deprotecting the thiol group.

According to a nineteenth aspect, the present disclosure provides a method of preparing a glycoglucagon comprising: a) providing a modified glucagon analogue comprising an additional cysteine residue substituted at position 25 of the glucagon peptide sequence; and b) reacting the thiol group of the cysteine residue with a glycosyl moiety substituted with a leaving group to provide a glycoglucagon. The glycosyl moiety may optionally be a sialylglycosyl moiety.

### BRIEF DESCRIPTION OF THE FIGURES

Exemplary embodiments of the present disclosure are described herein, by way of non-limiting example only, with reference to the following drawings.

FIG. 1. Scheme showing the process for preparing non-glycoinsulin, an insulin derivative which is not glycosylated.

FIG. 2. Scheme showing the process for glycosylating the non-glycoinsulin prepared by the scheme of FIG. 1.

FIG. 3. (A) Reversed-phase high-performance liquid chromatography monitoring of glycosylation reaction of FIG. 2 at (i) 2 h, (ii) 8 h, (iii) 24 h, and (iv) RP-HPLC of purified glycoinsulin, and (B) Matrix Assisted Laser Desorption/Ionisation Time of Flight Mass Spectrometry (MALDI-TOF MS) of (i) thiol insulin, (ii) glycoinsulin) and (iii) insulin dimer.

FIG. 4. Binding of insulin, non-glycoinsulin and glycoinsulin to (A) insulin receptor A and (B) insulin receptor B.

FIG. 5. Insulin tolerance test results showing changes in plasma glucose following administration of (A) insulin, non-glycoinsulin, glycoinsulin and glargine by intraperitoneal injection and (B) insulin, non-glycoinsulin and glycoinsulin by subcutaneous injection.

FIG. 6. Atomic force microscopy images of (A) 50 μM insulin solution (B) 200 μM insulin solution and (C) 200 μM glycoinsulin solution after 6 hours and 8 hours.

FIG. 7. Circular dichroism (CD) spectra of insulin and glycoinsulin in phosphate buffer (pH 7.5) at 25° C.

FIG. 8. Serum stability assay results of insulin and glycoinsulin at 37° C.

FIG. 9. Structures of (A) native glucagon and (B) glycosylated glucagon.

FIG. 10. cAMP activity assays of glucagon and glycoglucagon in HEK293AWT cells transiently transfected with pcDNA3-hGCGR. The data is normalised to maximum response of FSK (10 uM) control.

FIG. 11. Representative atomic force microscopy height images of (A) Glucagon (1 mg/mL=287 mM) and (B) Glycoglucagon (287 mM) solution incubated at 37° C. at day 4.

### DETAILED DESCRIPTION

Throughout this specification, unless the context requires otherwise, the word “comprise”, or variations such as “comprises” or “comprising”, will be understood to imply the inclusion of a stated step or element or integer or group of steps or elements or integers, but not the exclusion of any other step or element or integer or group of elements or integers. Thus, in the context of this specification, the term “comprising” means “including principally, but not necessarily solely”.

In the context of this specification, the term “about” is understood to refer to a range of numbers that a person of skill in the art would consider equivalent to the recited value in the context of achieving the same function or result.

In the context of this specification, the terms “a” and “an” refer to one or to more than one (i.e. to at least one) of the grammatical object of the article. By way of example, “an element” means one element or more than one element.

The term “subject” as used herein refers to mammals and includes humans, primates, livestock animals (e.g. sheep, pigs, cattle, horses, donkeys), laboratory test animals (e.g. mice, rabbits, rats, guinea pigs), performance and show animals (e.g. horses, livestock, dogs, cats), companion animals (e.g. dogs, cats) and captive wild animals. In particular embodiments, the subject is a human.

The term “peptide” means a polymer made up of amino acids linked together by peptide bonds. The terms “polypeptide” or “protein” may also be used to refer to such a polymer although in some instances a polypeptide or protein may be longer (i.e. composed of more amino acid residues) than a peptide. Typically, the term peptide is used to define a sequence of amino acids of up to about 70 amino acids. The term “protein” may typically be used to define a sequence of amino acids of greater than about 70 amino acids. Nevertheless, as will be familiar to a skilled person in the art, the terms may be used interchangeably herein and readily understood from context of their use.

An aspect of the present disclosure provides a method for glycosylating a peptide or protein, comprising the steps of: a) providing a peptide or protein comprising an amino acid residue comprising an unprotected nucleophilic group; and b) reacting the nucleophilic group with a glycosyl moiety substituted with a leaving group to provide a glycosylated peptide or protein.

The peptide or protein to which the method is applied may be any peptide or protein which it is desirable to glycosylate and which is amenable to glycosylation. In particular the peptide or protein is one which is subject to fibrillation. In exemplary embodiments described herein the peptide or protein may be insulin or glucagon. The A chain of the insulin peptide may comprise the amino acid sequence set forth in SEQ ID NO:4, or a variant thereof. The B chain of the insulin peptide may comprise the amino acid sequence set forth in SEQ ID NO:2, or a variant thereof. The glucagon may comprise the amino acid sequence set forth in SEQ ID NO:5, or a variant thereof.

Insulin is a heterodimeric peptide (A- and B-chain) with six cysteine residues that form a stable network of three disulfide bridges. As exemplified herein, and in accordance with embodiments of the present disclosure, insulin was modified by introduction of an additional cysteine residue at the N-terminal of the B-chain to provide a modified thiol insulin. The thiol insulin was reacted with bromo-acetamidyl undecasaccharide derived from human-type sialyloligosaccharide isolated from egg yolks via S_{N}2 chemistry to provide a mono-labelled disialo-insulin (termed herein “glycoinsulin”) at high yield, with high product homogeneity. The N-terminal of the B-chain of insulin was selected as the glycosylation site for two reasons: (i) it tolerates modification without a significant loss of activity; and (ii) N-terminal functionalisation of recombinant proinsulin (the single-chain precursor to mature insulin analogues) is amenable to scale-up.

The resulting glycoinsulin showed a near-native binding affinity for insulin receptors A and B in vitro and high glucose-lowering effects in vitro, irrespective of the route of administration (subcutaneous versus intraperitoneal). The glycoinsulin retained insulin-like helical structure and exhibited improved stability in human serum. Importantly, the glycoinsulin did not form fibrils at both high concentration and high temperature. The glycoinsulin produced may be used in various applications in place of native insulin, and to particular advantage in insulin pumps, which are particularly susceptible to problems caused by fibrillation of insulin.

As further exemplified herein, and in accordance with embodiments of the present disclosure, glucagon was modified by substitution of the tryptophan 25 residue with a cysteine residue to provide a modified glucagon. The modified glucagon was reacted with bromo-acetamidyl undecasaccharide derived from human-type sialyloligosaccharide isolated from egg yolks via S_{N}2 chemistry to provide a mono-labelled disialo-glucagon (termed herein “glycoglucagon”) at high yield and with high product homogeneity. The prepared glycoglucagon retained biological activity at the glucagon receptor relative to native glucagon, and showed significantly reduced fibrillation propensity.

Accordingly, particular embodiments of the present disclosure provide a method for preparing a derivative of a peptide or protein comprising a derivative moiety, comprising the steps of: a) providing a peptide or protein comprising an amino acid residue comprising an unprotected nucleophilic group; and b) reacting the nucleophilic group with a derivative moiety substituted with a leaving group to provide a derivative of the peptide or protein. A derivative of a peptide or protein comprising a derivative moiety may be referred to herein as a “derivatised” peptide or protein. In particularly preferred embodiments, the method does not involve the use of an enzyme.

In accordance with the Examples of the present application, particular embodiments of the present disclosure provide a method for glycosylating a peptide or protein, comprising the steps of: a) providing a peptide or protein comprising an amino acid residue comprising an unprotected nucleophilic group; and b) reacting the nucleophilic group with a glycosyl moiety substituted with a leaving group to provide a glycosylated peptide or protein. In particularly preferred embodiments, the method does not involve the use of an enzyme.

In some embodiments, the peptide or protein for use in step a) is modified from the native peptide or protein by introduction of the amino acid residue comprising an unprotected nucleophilic group as an additional residue or an amino acid substitution. Accordingly, the present disclosure further provides a method for preparing a derivative of a peptide or protein comprising a derivative moiety, comprising the steps of: a) modifying a peptide or protein to comprise an additional or substituted amino acid residue, wherein the amino acid residue comprises an unprotected nucleophilic group; and b) reacting the nucleophilic group with a derivative moiety substituted with a leaving group to provide a derivative of the peptide or protein. According to particular embodiments, the present disclosure further provides a method for glycosylating a peptide or protein, comprising the steps of: a) modifying a peptide or protein to comprise an additional or substituted amino acid residue, wherein the amino acid residue comprises an unprotected nucleophilic group; and b) reacting the nucleophilic group with a glycosyl moiety substituted with a leaving group to provide a glycosylated peptide or protein. The present disclosure further provides a method for preparing a derivative of a peptide or protein comprising a derivative moiety, comprising the steps of: a) providing a modified peptide or protein comprising an additional or substituted amino acid residue relative to the native peptide or protein, wherein the amino acid residue comprises an unprotected nucleophilic group; and b) reacting the nucleophilic group with a derivative moiety substituted with a leaving group to provide a derivative of the peptide or protein. The present disclosure further provides a method for glycosylating a peptide or protein, comprising the steps of: a) providing a modified peptide or protein comprising an additional or substituted amino acid residue relative to the native peptide or protein, wherein the amino acid residue comprises an unprotected nucleophilic group; and b) reacting the nucleophilic group with a glycosyl moiety substituted with a leaving group to provide a glycosylated peptide or protein.

The present inventors have surprisingly found that such methods of derivatisation, in particular glycosylation, can be used to prepare glycosylated peptides or proteins in an efficient, reproducible manner, with a high yield of the desired product and with high homogeneity with regards to the site of glycosylation. This is in advantageous contrast to existing enzymatic glycosylation methods, which typically lead to heterogenous products of glycosylation at various sites. Glycosylated proteins resulting from methods according to embodiments of the present disclosure were found to maintain desirable biological activity whilst demonstrating reduced or no fibril formation. In particular embodiments wherein the protein or peptide is insulin, glycosylated insulin was found to maintain its native structural folds and desired biological activity, whilst being resistant to the formation of fibrils at high temperature and high concentration and having enhanced stability in human serum in vitro. Similarly, in particular embodiments wherein the protein or peptide is glucagon, glycosylated glucagon was also found to maintain desired biological activity whilst showing markedly reduced or even no fibrillation.

Disclosed herein are methods for glycosylating a peptide or protein. Glycosylation refers to the introduction of a glycosyl (sugar) moiety covalently bonded to a peptide or protein. Glycosylation has been shown to advantageously inhibit fibrillation of a peptide or protein, at high temperature and high concentration, whilst maintaining desirable biological activity.

In order to prepare a derivative of a peptide or protein, such as glycosylate a peptide or protein in accordance with an embodiment of the method described herein, a modified version of the peptide or protein comprising an additional or substituted amino acid residue is typically first provided. The term “modified” as used herein in the context of a modified version of the peptide or protein (also referred to as a modified peptide or protein) means a peptide or protein that differs from the peptide or protein in its unmodified state, such as that which it is desired to derivatise (i.e. introduce a derivative moiety) for example glycosylate, at one or more amino acid positions of such unmodified peptide or protein. The peptide or protein, in its unmodified state, may be a naturally occurring or native peptide or protein, or may be a derivative, variant or fragment thereof.

An “additional” amino acid residue is an amino acid residue present in addition to the unmodified amino acid sequence of the peptide or protein, for example the native amino acid sequence of the peptide or protein. The additional amino acid residue may be located at a terminus of the peptide or protein, i.e. the N terminal or the C terminal, or at some other point along the amino acid sequence of the protein or peptide. In particular embodiments, the additional amino acid residue is present at the N-terminal of the peptide sequence. In some embodiments wherein the peptide is insulin or an analogue thereof, the additional amino acid residue is located on the A-chain of the insulin, such as at the N-terminal of the A-chain. In alternative embodiments wherein the peptide is insulin or an analogue thereof, the additional amino acid residue is located on the B-chain. In some preferred embodiments, the additional amino acid residue is located at the N-terminal of the B-chain, as exemplified herein; such embodiments are advantageous since addition of an amino acid at the N-terminal of the B-chain facilitates scale up of the synthesis of the modified B-chain or entire insulin, in particular because addition of the amino acid at the N-terminal of the B chain will not be affected by cleavage by the enzymes which cleave the C-peptide of pro-insulin from the B-chain at the C-terminus of the B-chain, and from the A-chain at the N-terminus of the A chain, thus allowing programing of recombinant DNA production/expression of a Cys-B-chain-C-peptide-A-chain prohormone as a means of synthesis. A “substituted” amino acid is present in place of an amino acid residue present in the native, or unmodified peptide sequence. In particular embodiments wherein the peptide or protein is glucagon, the substituted amino acid may replace the tryptophan residue at position 25 in native human glucagon.

A modified version of the peptide or protein, in the context of the present disclosure, need not be physically constructed or generated from the unmodified, for example, naturally occurring or native sequence, but may be chemically synthesised such that the sequence is “derived” from the unmodified sequence in that it shares sequence homology and function with the naturally occurring or native sequence. That is, rather than starting from the unmodified peptide or protein, a modified version of the protein or peptide may instead be synthesised to include the additional or substituted amino acid ab initio. The terms “naturally occurring” and “native” refer to peptides and proteins as encoded by and produced from the genome of an organism.

In the context of the present disclosure, the modified version of the protein or peptide may also be referred to as an analogue of the peptide or protein, as is appropriate. The skilled person will understand an analogue of a peptide or protein to refer to a peptide sequence having an additional or substituted amino acid, whilst substantially maintaining the functionality of the “starting” peptide or protein.

In the present disclosure, reference to a named peptide or protein may be understood as including its derivatives, variants and/or functional fragments, unless specified otherwise or suggested otherwise by context.

As used herein, the term “derivative” is intended to encompass chemical modification to a peptide or protein or more amino acid residues of a peptide or protein, including chemical modification in vitro, for example by introducing a group in a side chain in one or more positions of a peptide, such as a nitro group in a tyrosine residue or iodine in a tyrosine residue, by conversion of a free carboxylic group to an ester group or to an amide group, by converting an amino group to an amide by acylation, by acylating a hydroxy group rendering an ester, by alkylation of a primary amine rendering a secondary amine, or linkage of a hydrophilic moiety to an amino acid side chain. Other derivatives may be obtained by oxidation or reduction of the side-chains of the amino acid residues in the peptide. Modification of an amino acid may also include derivation of an amino acid by the addition and/or removal of chemical groups to/from the amino acid, and may include substitution of an amino acid with an amino acid analog (such as a phosphorylated amino acid) or a non-naturally occurring amino acid such as a N-alkylated amino acid (e.g. N-methyl amino acid), D-amino acid, β-amino acid or γ-amino acid. Particular derivatives as described in the context of the present disclosure refer to derivatives obtained by attachment of a derivative moiety, i.e. a substituent group, to a peptide or protein.

A variant of a peptide, such as a naturally occurring peptide or protein, in the context of the present specification, is a peptide or protein wherein one or more amino acids of the peptide sequence have been substituted for a different amino acid. In some embodiments, the protein or peptide to be glycosylated is a conservative variant of a naturally occurring or native peptide or protein, meaning the peptide or protein comprises one or more conservative amino acid substitutions. A “conservative amino acid substitution” is one in which an amino acid residue is replaced with another residue having a chemically similar or derivatised side chain. Families of amino acid residues having similar side chains, for example, have been defined in the art. These families include, for example, amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). For example, the substitution of the neutral amino acid serine (S) for the similarly neutral amino acid threonine (T) would be a conservative amino acid substitution. Those skilled in the art will be able to determine suitable conservative amino acid substitutions that do not eliminate the functional properties of the peptide sequence required in the context of the present disclosure. In particular embodiments, the variant will possess at least about 80% identity to the sequence of which it is a variant. The sequence may be about 80%, 81%, 82%, 83%, 84%, 85%, 86%, 87%, 88%, 89%, 90%, 91%, 92%, 93%, 94%, 95%, 96%, 97%, 98% or 99% identical to the sequence of which it is a variant. Numerous means are available, and will be known, to those skilled in the art for determining sequence identity, for example computer programs that employ algorithms such as BLAST (Basic Local Alignment Search Tool, Altschul et al., 1993, J. Mol. Biol. 215:403-410).

As will be apparent to and readily understood by a skilled person in the art of peptides and protein, the terms “analogue”, “variant” and “derivative” may be used somewhat interchangeably, such as when referring to a peptide sequence which has undergone amino acid substitutions, including for other amino acids with side-chain substitutions. The meaning of the terms and the peptides to which they refer will be readily understood from the context of the specification.

Embodiments of the present disclosure also relate to the modification of functional fragments of peptides and proteins. As used herein, the term “functional fragment” refers to a fragment of a peptide or protein is a subsequence of the peptide that performs a similar function and retains substantially the same activity as the peptide or protein sequence from which the fragment is derived.

The peptide or protein, or modified version of the peptide or protein, for use in embodiments of the present disclosure, may be produced using any method known in the art, including synthetically or by recombinant techniques such as expression of nucleic acid constructs encoding the peptide or protein. For example, a peptide may be synthesised using solid phase peptide synthesis, for example the Fmoc-polyamide mode of solid-phase peptide synthesis, for example continuous flow Fmoc solid phase peptide synthesis. Other synthesis methods include solid phase t-Boc synthesis and liquid phase synthesis. Purification can be performed by any one, or a combination of, techniques such as re-crystallisation, size exclusion chromatography, ion-exchange chromatography, hydrophobic interaction chromatography and reverse-phase high performance liquid chromatography using, for example, acetonitrile/water gradient separation.

The peptide or protein may be any protein or peptide which it is desirable to glycosylate. In some embodiments, the protein or peptide is selected from insulin, glucagon, glucagon-like peptide-1 (GLP1), amyloid β, α-synuclein, ataxin, F-box protein 7 (FBOXO7), prion protein (PrPsc), tau (hyperphosphorylated), transactive response DNA binding protein 43 (TDP43), superoxide dismutase 1 (SOD1), and huntingtin (with polyQ tract>33 residues), or a fragment, derivative or variant thereof. In particular embodiments, the peptide or protein is selected from the group consisting of insulin, glucagon, glucagon-like peptide-1 (GLP1) and amyloid β, or a fragment, derivative or variant thereof, in particular insulin and glucagon or a fragment, derivative or variant thereof, in particular insulin and glucagon. In some embodiments, the peptide or protein is the A chain or B chain of insulin alone; the derivative of the A or B chain, for example the glycosylated A or B chain may optionally subsequently be combined with the other chain. Where relevant, features disclosed herein in relation to insulin may also be applied to the A chain or B chain of insulin alone.

In some embodiments, the peptide may be insulin, or a fragment, derivative or variant thereof. Insulin finds particular use in embodiments of the invention, as it is prone to fibrillation, which can cause particular problems in users of insulin pumps by hindering ready flow of insulin through the pump system and to a user. Insulin thus benefits greatly from the glycosylation method of the present disclosure, since, as demonstrated by the Examples herein below, glycosylation is a means of reducing fibrillation at high concentration and high temperature, whilst maintaining a useful binding affinity for insulin receptors A and B, maintaining glucose-lowering effects, retaining helical structure following glycosylation and exhibiting improved stability in human serum. It is advantageous to provide one or more of these benefits to a peptide or protein glycosylated by an embodiment of the method of the present disclosure. Throughout the present specification, insulin glycosylated as described herein may alternatively be referred to as “glycoinsulin”.

In some embodiments wherein the peptide is insulin, the A chain of insulin is glycosylated. hi some embodiments wherein the peptide is insulin, the B chain of insulin is glycosylated. In particular embodiments, the N terminal of the A or B chain is glycosylated. The A or B chain may optionally be glycosylated by introduction of an additional amino residue, such as a cysteine residue, as described herein. In some embodiments, the B chain of insulin is glycosylated at the K29 position.

In some alternative embodiments, the peptide or protein is glucagon, or a fragment, derivative or variant thereof. Glucagon is also prone to forming amyloid-like fibril structures at the acidic pH required for solubility, and is therefore typically formulated as a lyophilised powder. Glucagon also forms fibrils at physiological pH, which has detrimental effects on its efficacy as a drug, such as for the treatment of hypoglycaemia, and suffers from limited aqueous solubility. Glucagon thus also benefits greatly from the glycosylation method of the present disclosure, since, as demonstrated by the Examples herein below, glycosylation is a means of reducing fibrillation of glucagon relative to native human glucagon, whilst maintaining useful biological activity. Alternatively or in addition, derivatisation may be used to enhance solubility of glucagon and other peptides. Throughout the present specification, glucagon glycosylated as described herein may alternatively be referred to as “glycoglucagon”.

Both insulin and glucagon also find use in methods of derivatisation with a derivative moiety other than a glycosyl moiety as described herein.

The peptide or protein for use in accordance with methods of derivatisation, for example glycosylation described herein comprises an amino acid residue comprising an unprotected nucleophilic group, optionally incorporated into the peptide or protein as an additional or substituted amino acid residue. Preferred unprotected nucleophilic groups are effective nucleophiles in S_{N}2 reactions, preferably more nucleophilic in S_{N}2 reactions than other nucleophilic groups present in the peptide or protein. An exemplary nucleophilic group which is particularly suitable for use in the present invention is a thiol group. A thiol group may be provided by use of a cysteine residue. Other unprotected nucleophilic groups which may be employed in accordance with the present disclosure include an imidazole group (for example present in a histidine residue), an amine group (for example present in a lysine residue), an amide group (for example present in an asparagine residue) and an alcohol group (for example present in a serine residue). In some particular embodiments, the nucleophilic group is an amine group, for example present in a lysine residue, for example at the K19 position of the B chain of insulin. Natural amino acid residues, such as cysteine, are advantageous since they can be readily engineered into a genetic code for scale up of peptide synthesis. The unprotected nucleophilic group may be present before the amino acid residue is incorporated into the peptide or protein, or may be introduced after the amino acid is incorporated into the peptide or protein. In some embodiments, the nucleophilic group may be in a protected form when the amino acid is incorporated into the peptide or protein, and subsequently deprotected.

The unprotected nucleophilic group of the amino acid is reacted with a derivative moiety substituted with a leaving group, such as a glycosyl moiety substituted with a leaving group to provide a derivative of a peptide or protein comprising a derivative moiety, such as a glycosylated peptide or protein. In particular embodiments, this reaction takes place via S_{N}2 chemistry. Typically, this reaction involves nucleophilic substitution of the leaving group by the nucleophilic group.

A glycosyl group is a saccharide, i.e. sugar, substituent. The glycosyl moiety may be any glycosyl group which it is desirable to attach to a peptide or protein to provide a glycosylated peptide or protein. The glycosyl moiety may be a monosaccharide substituent, or an oligosaccharide or polysaccharide substituent made up of multiple monosaccharide units. The saccharide units of an oligosaccharide or polysaccharide substituent may be linear or branched. The terms “oligosaccharide” and “polysaccharide” encompass all simple to complex oligosaccharides and polysaccharides.

In some preferred embodiments, the glycosyl moiety does not comprise glucose; such embodiments are advantageous since, upon degradation of the glycosylated peptide or protein, no glucose is released, which could otherwise raise blood sugar levels of a subject to which the glycosylated protein or peptide is administered. It is particularly desirable that the glycosyl moiety does not comprise glucose when the peptide or protein is insulin, or a derivative, analogue variant or functional fragment thereof, since, upon administration of the glycoinsulin to a diabetic patient experiencing high blood sugar levels, any blood sugar increases due to degradation of the glycoinsulin and release of glucose would further exacerbate the situation.

In some preferred embodiments, the glycosyl moiety is a sialylglycosyl moiety. A ‘sialylglycosyl’ moiety is a glycosyl group containing a sialic acid unit. Every monosaccharide unit of the sialylglycosyl moiety need not be a sialyl unit. The sialylglycosyl moiety may be, for example, a sialyloligosaccharide moiety. For example, the sialylglycosyl moiety may be a sialyl undecasaccharide moiety. A sialylglycosyl moiety may advantageously prolong the half-life of a peptide or protein in vivo, permitting, for example, a reduction of the frequency of administration. Without wishing to be bound by theory, it is thought that the use of a sialylglycosyl moiety increases the half-life of the glycosylated protein or peptide by inhibiting liver-mediated decomposition of the protein or peptide. Throughout this specification, the term “sialo” may be used in place of “sialyl”.

Alternative derivative moieties which may be used in methods of the present disclosure include fatty acid or polyethylene glycol groups, or a further unit of the same peptide which may be attached to the protein or peptide through disulfide, thiol-maleimide or thioether chemistry, such as to improve the in vivo half-life of insulin and analogues for better and efficient treatment of diabetes.

Further possible derivative moieties which may be attached to proteins or peptides by methods of the present disclosure include glucose-sensing units, such as phenylboronic acid (‘PBA’). Derivatives of insulin incorporating a glucose-sensing unit are especially advantageous since they provide an inactive form of insulin which only becomes active upon glucose levels reaching a threshold so as to trigger the release of the glucose sensing unit such as PBA (wherein the glucose sensing unit such as PBA binds glucose) and free insulin.

Further possible derivative moieties which may be attached to proteins or peptides by methods of the present disclosure include blood-brain barrier transporter units (also known as blood brain barrier shuttles), such as apamin peptides and TAT (‘transactivator of transcription’, a peptide (GRKKRRQRRRPQ (SEQ ID NO:1)) derived from the HIV protein).

In some embodiments, proteins or peptides may be derivatised by methods of the present disclosure by attachment of further proteins of peptides as derivative moieties, for example further units of the same protein or peptide to prepare dimers, trimers, and other oligomers and polymers. In particular exemplary embodiments, insulin may be derivatised by attachment of further insulin units, to make, for example, an insulin dimer, trimer, hexamer or other oligomer or polymer of insulin. Analogous oligomers or polymers may be obtained using glucagon as the protein or peptide.

A skilled person will readily appreciate that other derivative moieties may also be used, including other pharmacophores. Precursors of the desired derivative moieties substituted with a leaving group may also be used, such that the product of the reaction of the nucleophilic group of the peptide or protein with the derivative moiety substituted with a leaving group must undergo further reaction or processing to arrive at the precise derivative desired.

In some alternative aspect and embodiments, reaction between an unprotected nucleophilic group and a derivative moiety takes place by a mechanism other than S_{N}2. Any suitable derivative moiety may be used which reacts with the unprotected nucleophilic group in order to introduce the derivative moiety onto the peptide or protein.

In some embodiments, the derivative moiety is introduced by way of disulfide, thiol-maleimide or thioether chemistry with the thiol group of a cysteine residue as mentioned above.

The leaving group may be any substituent which facilitates participation of the glycosyl moiety in a substitution reaction, i.e. a leaving group which is readily substituted by the nucleophile of the modified version of the peptide or protein. Suitable leaving groups will be readily determined by and well within the understanding of a person skilled in the art of synthetic chemistry. Suitable leaving groups include iodide, bromide, chloride, tosyl, mesyl, and haloacetamidyl groups, for example bromoacetamidyl. For example, in embodiments where the glycosyl moiety is an oligosaccharide, and the leaving group is a bomoacetamidyl group, the glycosyl moiety attached to a leaving group is a bromo-acetamidyl oligosaccharide. For example, the glycosyl moiety attached to a leaving group may be a bromoacetamidyl undecasaccharide, such as a bromoacetamidyl sialyl undecasaccharide, such as of formula (I) shown below:

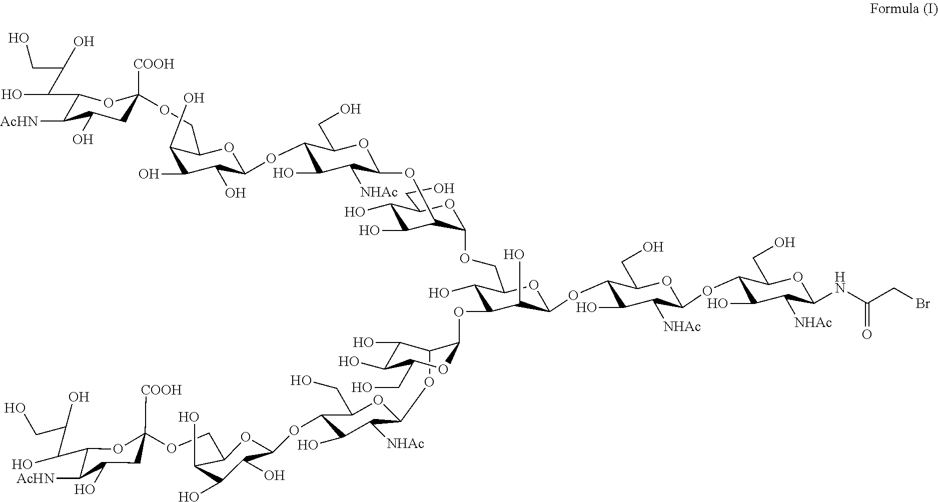

In some particular embodiments wherein the glycosyl moiety depicted in Formula (I) is used and the peptide is insulin, the glycosylated insulin produced has the structure of Formula (II) below:

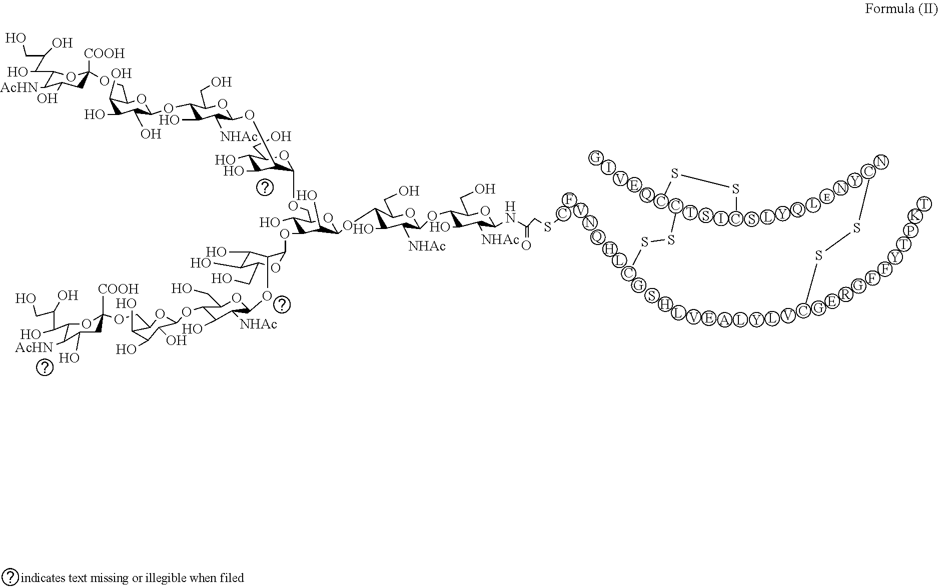

In some particular embodiments wherein the glycosyl moiety depicted in Formula (I) is used and the peptide is glucagon, the glycosylated glucagon produced has the structure of Formula (III) below:

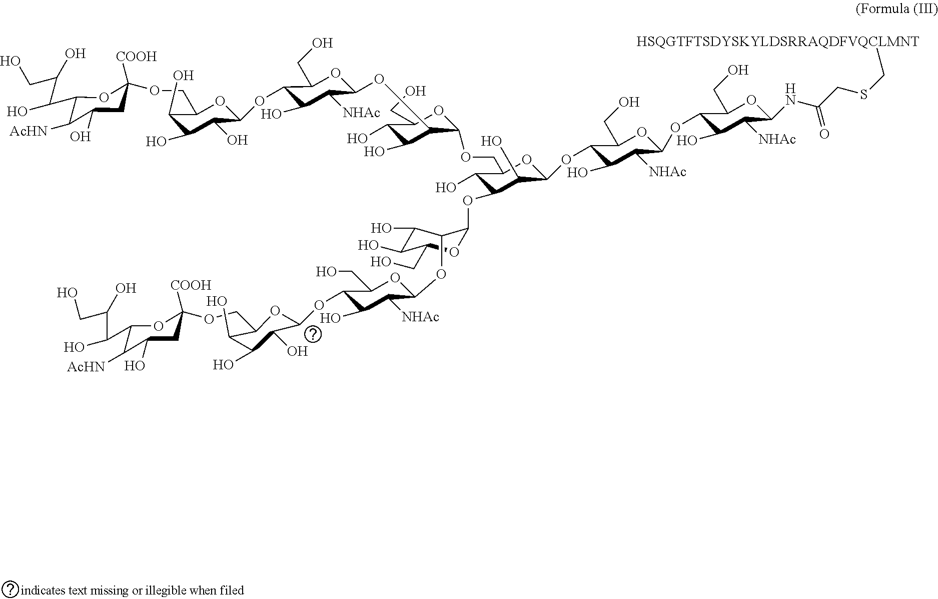

In some embodiments, the glycosyl moiety is esterified such that the leaving group is the conjugate base of an alcohol.

In particular embodiments, the nucleophilic group is a thiol group, such as provided by a cysteine residue, and the leaving group is a bromide. In some alternative embodiments, the nucleophilic group is an amine group, such as provided by a lysine residue, such as the K29 residue of the B chain of insulin, and the glycosyl group is esterified such that the leaving group is the conjugate base of an alcohol.

The step of modifying the peptide or protein by introducing an amino acid residue comprising an unprotected nucleophilic group as an additional or substituted amino acid residue, may comprise the following steps:
a1) protecting the nucleophilic group of the amino acid with a protecting group;
a2) preparing a modified version of the protein or peptide comprising the amino acid, wherein the nucleophilic group of the non-native amino acid residue is protected; and then
a3) deprotecting the nucleophilic group.

The protecting group may be applied to the nucleophilic group, for example, prior to incorporation of the amino acid into the peptide sequence or part of the peptide sequence of the peptide or protein, applied to the nucleophilic group following incorporation of the amino acid into part of the peptide sequence of the peptide or protein but before the full sequence is prepared, or applied to the nucleophilic group following preparation of the peptide sequence but before the further structure of the protein is established, for example before the creation of disulphide bonds between chains of a protein structure.

By way of example, in the case of a thiol nucleophilic group, a tert-butyl protecting group may be used. Other appropriate protecting groups and methods will be readily apparent to a skilled person.

The steps of protecting the nucleophilic group prior to preparation of the modified version of the protein or peptide, for example by way of solid-phase peptide synthesis, followed by deprotecting the nucleophilic group is particularly advantageous where the nature of the nucleophilic group and the peptide or protein is such that the nucleophilic group may otherwise interfere in the assembly of the protein or peptide, such as the folding of the protein or peptide. For example, in the case of insulin, in particular when a cysteine residue is used as the additional or substituted amino acid having a nucleophilic group, the protection and deprotection of the thiol group facilitates assembly of the insulin without the risk of unwanted disulphide shuffling between the cysteine group and the two cystine groups present in the insulin structure during assembly of the protein and formation of the disulphide bonds. Once the structure of the protein is prepared, the protecting group is removed from the nucleophilic group, and the resulting modified insulin is stable and the disulphide bonds do not scramble. The modified version of the insulin wherein the nucleophilic group of the non-native amino acid residue is protected may have the structure of formula (IV) below:

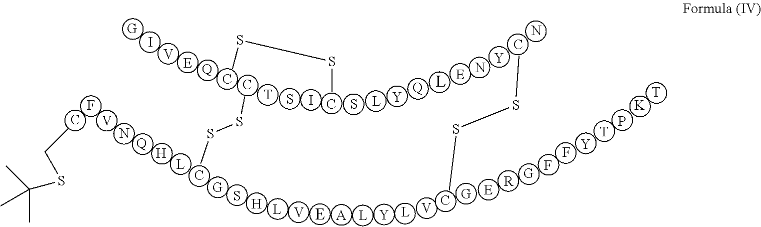

An advantage associated with embodiments of the method disclosed herein is that a derivatised peptide or protein, such as a glycosylated peptide or protein can be obtained in high yield, and with high specificity with regard to the site of derivatisation (i.e. homogeneity of derivatisation, for example glycosylation site and hence homogeneity of the resulting derivatised product). Accordingly, the present disclosure provides a method wherein at least 50%, at least 60%, at least 70%, at least 80%, at least 90%, at least 95%, at least 96%, at least 97%, at least 98%, at least 99%, such as about 100% of the produced derivatised peptide or protein is derivatised, for example glycosylated, in the same position on the peptide or protein. In particular embodiments, almost all, i.e. about 100%, of the produced derivatised peptide or protein is derivatised, for example glycosylated, in a single position on the peptide or protein.

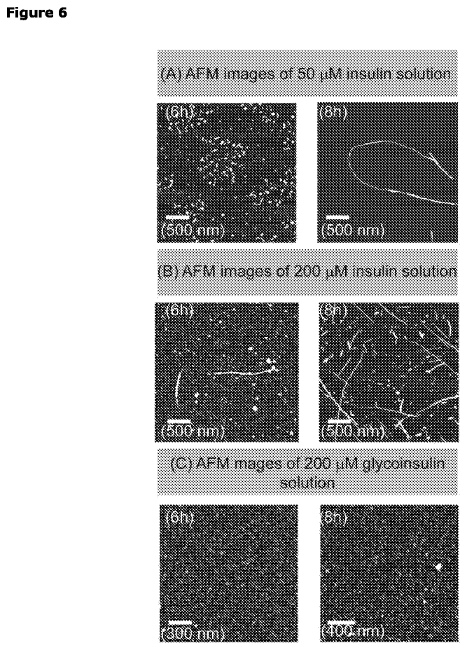

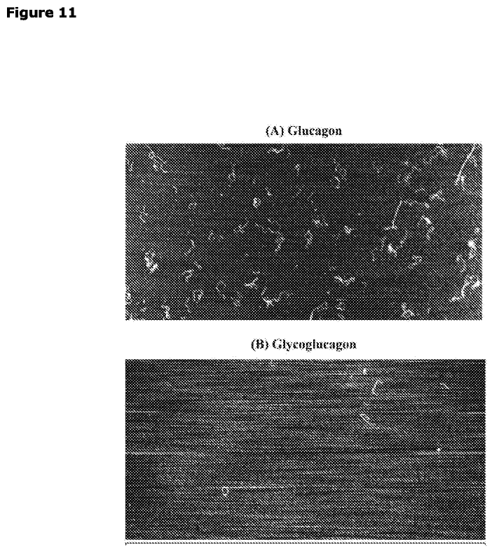

Also provided herein is a method of inhibiting fibrillation of a peptide or protein, comprising any of the methods of glycosylation described herein, as well as glycosylated peptides or proteins obtained by such methods. Inhibition of fibrillation can be assessed, for example, by use of atomic force microscopy (AFM) to visualise fibrillation of a peptide or protein under various conditions, including, for example, high temperature or concentration. Such use of AFM to visualise a reduction in fibrillation following glycosylation of insulin according to a method of an embodiment of the present disclosure is exemplified in Example 5, and the results shown in FIG. 6. Use of AFM to visualise a reduction in fibrillation following glycosylation of glucagon according to a method of an embodiment of the present disclosure is exemplified in Example 11, and the results shown in FIG. 11.

Also provided herein are methods of enhancing solubility of a peptide or protein, comprising a method of derivatisation, for example glycosylation, as described herein.

The present disclosure further provides a derivatised peptide or protein, such as a glycosylated peptide or protein, obtained or obtainable by a method as described herein. The present disclosure further provides a glycosylated peptide or protein, such as a glycosylated insulin or glucagon, obtained or obtainable by a method as described herein, having a reduced tendency to form fibrils relative to the native peptide or protein.

The present disclosure further relates to sialylglycosylated insulin, or an analogue, derivative, variant or functional fragment thereof, and, in particular embodiments, wherein the sialylglycosylated insulin has a reduced tendency to fibrillate relative to native insulin.

The present disclosure further relates to sialylglycosylated glucagon, or an analogue, derivative, variant or functional fragment thereof, and, in particular embodiments, wherein the sialylglycosylated glucagon has a reduced tendency to fibrillate relative to native glucagon.

A further aspect of the present inventors' discovery is that an insulin variant comprising three disulphide bridges and a non-native amino acid residue having an unprotected nucleophilic group, such as a single, additional cysteine residue (without a protecting group present) is stable or substantially stable, i.e. does not undergo any substantial disulphide shuffling or other conformational changes which might otherwise impact its biological properties. In particular, this insulin analogue is stable in water, particularly at a pH of about 6. This stable insulin analogue can be readily prepared by methods of the present disclosure, and used for further derivatisation, such as glycosylation. Accordingly, the present disclosure also relates to insulin analogues comprising disulphide bridges and a non-native amino acid residue having an unprotected nucleophilic group, preferably an additional non-native amino acid residue having an unprotected nucleophilic group. In particular, the present disclosure relates to insulin analogues comprising disulphide bridges and at least a single cysteine residue. In some embodiments, the single cysteine residue is located on the A chain of insulin, for example the N terminal of the A chain. In some embodiments, the single cysteine residue, or other amino acid residue having an unprotected nucleophilic group, is located on the B chain of insulin. In particular embodiments, the cysteine, or other amino acid residue having an unprotected nucleophilic group, is located at the N-terminal of the B chain of insulin, as shown in Formula (V) below:

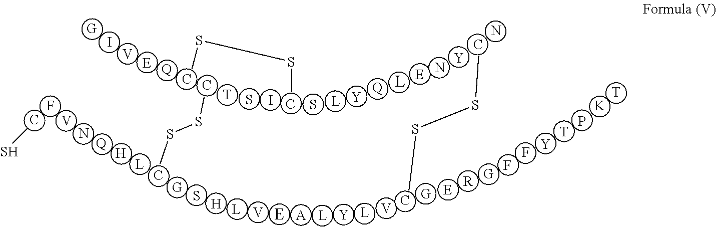

The insulin analogue preferably maintains the same three-dimensional conformation, or substantially the same three-dimensional conformation, as native insulin, for example maintains the folding structure of native insulin, and/or maintains the same biological activity, or substantially the same biological activity, as native insulin.

The present disclosure further relates to a glucagon analogue containing an additional or substituted amino acid residue having an unprotected nucleophilic group, for example a cysteine residue, for example as a substitution of the tryptophan at position 25 in native human glucagon.

The above insulin and glucagon analogues are advantageous in that they find potential use in large-scale production of glycoinsulin and glycoglucagon with reduced fibrillation propensity, via recombinant DNA methods and chemically directed glycosylation as described herein.

The discovery of the above insulin and glucagon analogues are also advantageous in that they find use in the production of other derivatives, such as by incorporation of the derivative moieties discussed above.

Accordingly, the present disclosure provides such insulin derivatives, and such corresponding glucagon derivatives.

The present disclosure further relates to use of the above insulin and glucagon analogues in a method of preparing glycoinsulin or another insulin derivative, or glycoglucagon or other glucagon derivative respectively, optionally according to a method as disclosed herein, and to methods of preparing an insulin derivative or glucagon derivative comprising reacting the above insulin analogue or glucagon analogue with a derivative moiety substituted with a leaving group, such as a glycosyl moiety substituted with a leaving group. In some embodiments, the derivative moiety substituted with a leaving group may be a fatty acid substituted with a leaving group, a polyethylene glycol substituted with a leaving group, a glucose-sensing unit substituted with a leaving group, or a blood-brain barrier transporting unit substituted with a leaving group. The derivative moiety substituted with a leaving group may be, for example, any of the aforementioned derivative moieties having a halogen substituent (e.g. having bromide or fluoride leaving groups) or a maleimide substituent. Embodiments wherein the derivative moiety is a blood-brain barrier transporting unit find particular use in the treatment or prevention of neurodegenerative disorders; defects in insulin signalling in the brain are thought to contribute to neurodegenerative disorders, which may be addressed by such derivatives.

The present disclosure further provides a peptide or protein derivative, such as a glycosylated peptide or protein, wherein the derivative moiety such as the glycosyl moiety is connected to the peptide or protein via an additional or substituted amino acid residue relative to the native peptide or protein. In some embodiments, the additional or substituted amino acid is cysteine. In some embodiments, the derivative, such as the glycosylated protein or peptide, has the same three-dimensional conformation, or substantially the same three-dimensional conformation, as the native peptide or protein, and/or maintains the same biological activity, or substantially the same biological activity, as the native peptide or protein. In some embodiments, the glycosyl moiety is a sialylglycosyl moiety. In alternative embodiments, the derivative moiety may be an alternative group to a glycosyl moiety as discussed above.

Also provided herein are methods of treating, managing or preventing a condition comprising administering to a patient in need thereof a peptide or protein derivative, such as a glycosylated peptide or protein, as described herein and/or obtained by a method as described herein. The method may be a method of treating or managing diabetes, a method of managing or lowering blood sugars, a method of managing or raising blood sugars, a method of treating or preventing hyperglycaemia, a method of treating or preventing hypoglycaemia, a method of treating or preventing a neurodegenerative disorder, a method of treating or managing an overdose of beta blockers or calcium channel blockers, a method of treating low blood pressure, a method of inhibiting gastrointestinal motility, for example to assist in radiologic examinations, and/or a method of diagnosing insulinoma. In particular embodiments wherein the peptide derivative is an insulin derivative, the method may be a method of treating or managing diabetes, a method of managing or lowering blood sugars, a method of treating or preventing hyperglycaemia, and/or a method of treating or preventing a neurodegenerative disorder. In particular embodiments wherein the peptide derivative is a glucagon derivative, the method may be a method of treating hypoglycaemia, a method of managing or raising blood sugars, a method of treating or managing an overdose of beta blockers or calcium channel blockers, a method of inhibiting gastrointestinal motility, for example to assist in radiologic examinations, and/or a method of diagnosing insulinoma. Also provided is use of a peptide or protein derivative, such as a glycosylated peptide or protein, according to the present disclosure, or obtained by a method of the present disclosure, in the manufacture of a medicament for treatment of such conditions.

It will be appreciated that features described herein in relation to one aspect of the present disclosure may be readily applied or modified for application to another aspect of the present disclosure. Disclosure of a feature in relation to one aspect includes its disclosure in relation to other aspects.

It will be appreciated by persons skilled in the art that numerous variations and/or modifications may be made to the invention without departing from the spirit or scope of the invention as broadly described. The present embodiments are, therefore, to be considered in all respects as illustrative and not restrictive.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

The present invention will now be further described in greater detail by reference to the following specific examples, which should not be construed as in any way limiting the scope of the invention.

### EXAMPLES

### Example 1

### Synthesis of Modified Insulin Comprising Additional Cysteine Residue

Insulin is a heterodimeric peptide (A- and B-chain) with six cysteine residues that form a stable network of three disulfide bridges (three cysteines). The N-terminal of the B-chain of insulin was selected as the glycosylation site for two reasons: (i) it tolerates modification without a significant loss of activity; and (ii) N-terminal functionalisation of recombinant proinsulin (the single-chain precursor to mature insulin analogues) is amenable to scale-up.

An additional cysteine residue was incorporated at the N-terminal of the B-chain of human insulin (SEQ ID NO:2) with temporary tert-butyl (tBu) side chain protection of the cysteine residue to avoid unwanted disulfide shuffling during stepwise folding. The use of an extra natural amino acid (cysteine residue) for functionalisation has the advantage that the synthesis of the modified insulin peptide may be scaled up, if necessary, by a recombinant DNA method. The resulting modified insulin B chain comprises the amino acid sequence set forth in SEQ ID NO:3.

Fmoc-protected L-α-amino acids and HCTU were purchased from GL Biochem (Shanghai, China). TentaGel R PHB-Thr (t-Bu) Fmoc (for the synthesis of B-chain) was purchased from Rapp Polymere (Tübingen, Germany) and Rink Amide MBHA LL resin (for the synthesis of A-chain) was purchased from NovaBiochem (subsidiary of Merck, Melbourne, Australia). TFA was obtained from Auspep (Melbourne, Australia). Acetonitrile, dichloromethane, diethyl ether, DMF, and methanol were from Merck (Melbourne, Australia). 2-pyridyl disulfide (DPDS) was purchased from Fluka (Buchs, Switzerland). Human serum (Sigma Lot No SLBX6020) and all other reagents were purchased from Sigma-Aldrich (Sydney, Australia).

### a) Peptide Synthesis

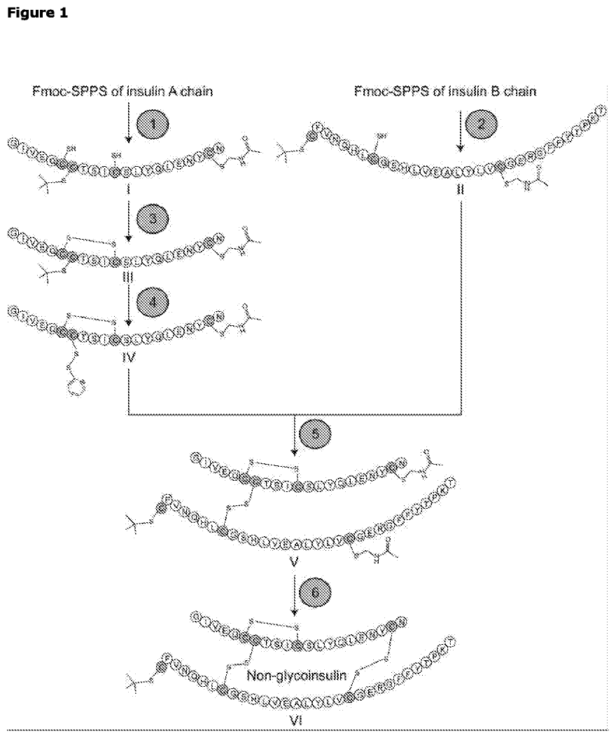

The A-chain (SEQ ID NO:4) and modified B-chain with the extra cysteine residue (SEQ ID NO:3) were prepared via continuous flow Fmoc (N-(9-fluorenyl)methoxycarbonyl) solid phase peptide synthesis. The two peptide chains (A and B, sequences shown in FIG. 1 as structures I and II) were assembled on preloaded (B chain) or Rink amide (A chain) resins using Fmoc solid phase synthesis on a microwave-assisted liberty peptide synthesiser (CEM Liberty, Mathew, USA).

The A-chain (I) was synthesised using the solid phase peptide synthesis (SPPS) method described above. Fmoc-Asp-OtBu was coupled on to the Rink amide resin (loading 0.36 mmol/g) via the side-chain on a 0.1 mmol scale. Cys(tBu) and Cys(Acm) were incorporated at position A7 and A20 respectively (see Step 1 of FIG. 1).

An extra amino acid, Cys(tBu), was incorporated at the N-terminal of the B-chain. The B-chain (II) of modified human insulin was assembled via microwave-assisted SPPS on a 0.1 mmol scale using preloaded FmocThr(tBu)-TentaGel resin (loading 0.18 mmol/g) and Cys(Acm) incorporated at position B19 (see Step 2 of FIG. 1).

The side chain-protecting groups of Fmoc-amino acids were TFA-labile, except for tBu-protected and Acm-protected cysteine (Cys) in the A-chain (A7 and A20 respectively) and Cys (Acm) in the B-chain (B19). The peptides were synthesised on a 0.1 scale using instrument default protocols with a 4-fold molar excess of Fmoc-protected amino acids (0.4 mmol) that were activated by using 3.8-fold excess of HCTU in the presence of excess of DIEA. Nα-Fmoc protecting groups were removed with piperidine/DMF (20% v/v). Coupling and deprotection steps on the synthesiser were carried out at 75° C. using 25 W microwave power for 5 min and 60 W microwave power for 5 or 3 min, respectively.

Analytical RP-HPLC analysis of the purified compounds was performed with two Waters RP-HPLC systems: Analytical HPLC Waters™ 600 coupled with a Waters™ 2487 detector and semi-preparative HPLC Waters™ 600 coupled with a Waters™ 996 detector. The RP-HPLC profiles were acquired using a Phenomenex Gemini C18 analytical column (4.6×250 mm, pore size 300 Å, particle size 5 μm) at a constant flow rate of 1.5 mL/min, in a gradient mode with buffer A, 0.1% aq. TFA, and buffer B, 0.1% TFA in acetonitrile, monitoring at a wavelength of 214 nm, which is characteristic for the amide bond. All RP-HPLC purifications were performed using a Phenomenex C18 preparative column (22×250 mm) in a gradient mode with eluent A, 0.1% aq. TFA, and eluent B, 0.1% TFA in acetonitrile.

MALDI TOF MS was carried out on a Bruker Ultraflex II instrument (Bruker Daltonics, Bremen, Germany) and used to characterise the peptides at each intermediate step using sinapinic acid (3,5-dimethoxy-4 hydroxycinnamic acid) as matrix. The matrix was made up in 75% acetonitrile containing 0.1% TFA.

### b) Synthesis of Insulin Analogue (Non-Glycoinsulin)

The A- and B-chain peptides synthesised above were then detached from the solid support by treating with a cleavage cocktail containing TFA/anisole/DODT/TIPS (94/2.5/1.5/1%) for 2 h at RT. The resin was filtered and washed with TFA. The filtrate was concentrated by evaporation under a stream of N2 and then precipitated with ice-cold diethyl ether to obtain the peptides as powders. The precipitate was centrifuged at 4000 rpm for 5 min and washed with ice cold diethyl ether five times. The peptides [Cys6,11(SH), Cys7(tBu), Cys20(Acm)] A-chain (I) and [Cys0(tBu), Cys7(SH), Cys19(Acm)] B-chain (II) were then freeze dried. The peptide purity was confirmed by analytical RP-HPLC and MALDI TOF MS (A-chain reduced form I m/z 2509.616; calcd. 2512.701; B-chain (II) m/z 3663.326; calcd. 3662.085).

The intramolecular disulphide bond of the A-chain was then formed (see Step 3 of FIG. 1). The crude [Cys6,11(SH), Cys7(tBu), Cys20(Acm)] A-chain (I) (120 mg, 47.8 μmol) was dissolved in 20% acetonitrile in water (total 120 mL). The DPDS solution (10.51 mg dissolved in 8 mL of methanol) was added to the magnetically stirred peptide solution. This reaction was complete at 30 min at RT as monitored by analytical RP-HPLC. The solution was loaded onto a preparative RP-HPLC column through the C-line for purification and freeze dried to give pure Cys6-Cys11 intramolecularly disulfide-bonded insulin A-chain, [Cys7(tBu), Cys20(Acm)] (III) (34.2 mg, 13.62 μmol, yield 28.5%). MALDI-TOF MS showed a single peak for [Cys7(tBu), Cys20(Acm)] (III) at m/z 2508.88, calcd. 2510.70.

Cys7(tBu) of the A-chain was then converted to Cys7(Pyr) (see Step 4 of FIG. 1). The purified [Cys7(tBu), Cys20(Acm)] A-chain (III) (30.0 mg, 11.94 μmol) and 4 equivalents of DPDS (11.52 mg, 47.76 μmol) were dissolved in 0.78 mL TFA and anisole (9:1 v/v) in an ice bath. Then, 0.78 mL of TFMSA/TFA (1:4 v/v) was added to the magnetically stirred mixture, and stirring was continued for 45 min at 0° C. The peptide was collected by precipitation with ice cold diethyl ether followed by centrifugation, and purified by using preparative RP-HPLC. The pure A-chain [Cys7(Pyr), Cys20(Acm)] (IV) (12.3 mg, 4.79 μmol, yield 40%) was obtained after freeze-drying, and identified by MALDI-TOF MS (m/z 2564.23, calcd. 2563.85).

The A-chain was then combined with the modified B-chain (see step 5 of FIG. 1). The purified A-chain [Cys7(Pyr), Cys20(Acm)] (IV) (10 mg, 3.9 μmol) was dissolved in 1 mL of 6 M GnHCl (0.1 M Gly/NaOH) buffer (pH 8.5). The [Cys0 (tBu), Cys7(SH), Cys19(Acm)] B-chain (II) (17 mg, 4.64 μmol was dissolved in 1 mL of deionised water and added slowly to the magnetically stirred A-chain solution. This reaction was complete in 20 min at RT. The resulting peptide, [A Cys20(Acm)-B Cys19(Acm)] (V), was purified by using preparative RP-HPLC and freeze-dried to obtain 14 mg (2.29 μmol, yield 49% calculated from the B-chain). The peptide (V) was identified by MALDI-TOF MS (m/z 6113.97, calcd. 6112.94).

Finally, iodine oxidation was used to generate non-glycoinsulin ((VI) of FIG. 1, step 6 of FIG. 1). The A-B peptide [A Cys20(Acm)-B Cys19(Acm)] (V) (11 mg, 1.8 μmol) was dissolved in a solvent mixture of 5.6 mL glacial acetic acid, 0.27 mL of 60 mM HCl. The dissolved peptide solution was then treated with iodine/acetic acid (20 mM, 7.5 mL) to remove Acm groups. This iodine oxidation was stopped by adding ice cold diethyl ether after 45 min at RT. Then, the peptide was purified by preparative RP-HPLC and freeze-dried to obtain 2.1 mg (0.351 μmol, yield 19.5%) of Cys0(tBu) insulin analogue, non-glycoinsulin (VI). The overall yield of non-glycoinsulin (VI) calculated from the B-chain was ˜7%. The purity of the peptide was confirmed by analytical RP-HPLC and the mass was confirmed by MALDI-TOF MS (VI, m/z 5969.34, calcd. 5970.94).

### Example 2

### Glycosylation

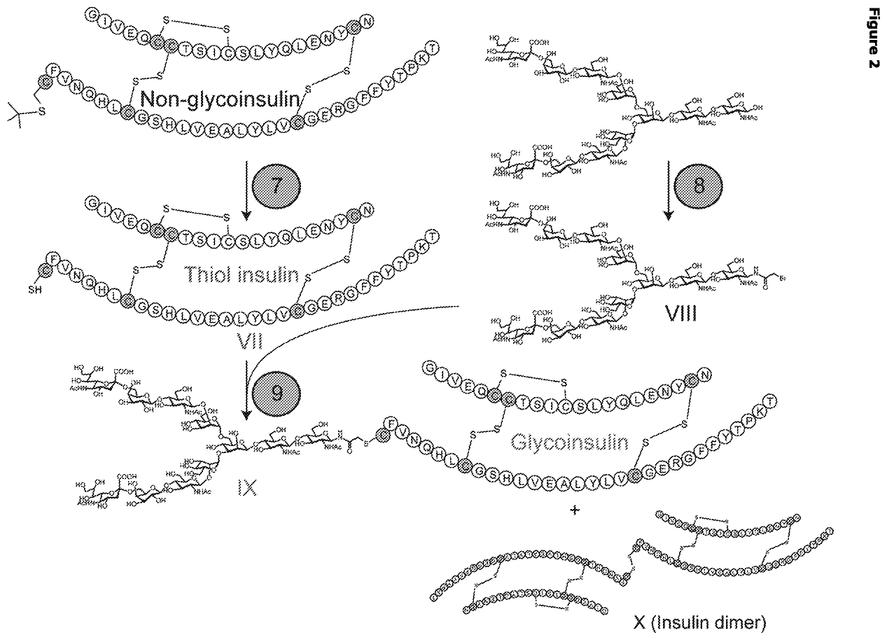

Glycosylation of the Cys0(tBu) insulin analogue prepared in Example 1 is shown in FIG. 2. The product of the described glycosylation is hereinafter referred to as “glycoinsulin”.

### a) Preparation of Thiol Insulin, a Starting Material for Glycosylation (See Step 7 of FIG. 2)

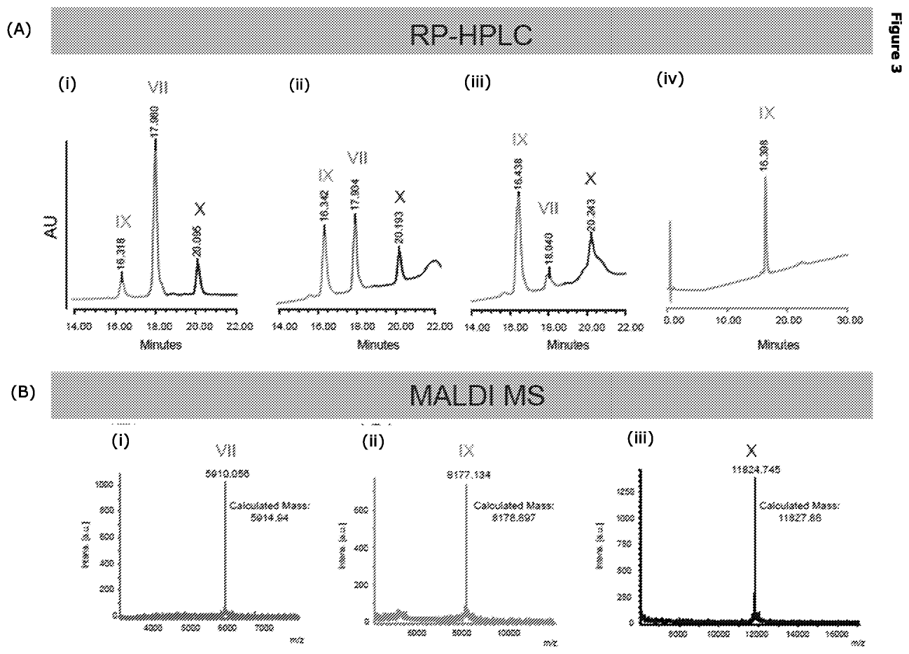

The purified Cys0(tBu) insulin analogue (non-glycoinsulin; VI in FIG. 1) (1.8 mg, 0.3 μmol), was dissolved in 0.2 mL TFA and anisole (9:1 v/v) in an ice bath. Then, 0.2 mL of TFMSA/TFA (1:4 v/v) was added to peptide solution (30 min at 0° C.). Ice cold ether was added after 30 min to precipitate the peptides. The resulting thiol insulin (VII in FIG. 2) was purified by preparative RP-HPLC. The reaction time was able to be reduced to 3 min instead of 30 min when a small-scale reaction was carried out (0.4 mg in 5 μL TFA/anisole (9:1) and 5 μL TFMSA/TFA (1:4)). For this small scale highly concentrated reaction, 3 minutes was enough to obtain the desired product with similar yield. The reaction was monitored to avoid side reactions. The purity of thiol insulin (VII) was confirmed by analytical RP-HPLC and the mass was confirmed by MALDI-TOF MS, as shown in FIG. 3 (m/z 5910.06, calcd. 5914.94). The yield of this reaction was found to be ˜68% (1.2 mg of VII; 0.203 μmol.

b) Extraction of Sugars from Egg Yolks and Conversion to Br-Derivative (See Step 8 of FIG. 2)

The human type complex disialo-oligosaccharide was isolated from egg yolks (as described in Kajihara, Y.; Suzuki, Y.; Yamamoto, N.; Sasaki, K.; Sakakibara, T.; Juneja, L. R., Prompt chemoenzymatic synthesis of diverse complex-type oligosaccharides and its application to the solid-phase synthesis of a glycopeptide with Asn-linked sialyl-undeca- and asialo-nonasaccharides. Chemistry 2004, 10 (4), 971-85) and derivatised to form bromo-acetamidyl undecasaccharide (VIII, FIG. 2 Step 8) by the method reported in Murase, T et al., “Efficient and systematic synthesis of a small glycoconjugate library having human complex type oligosaccharides”, Carbohydr Res 2009, 344 (6), 762-70.

### c) Glycosylation of Insulin

This reaction was carried out in Milli-Q water (pH 5-6) in which both the peptide and sugars were soluble. The non-glycoinsulin peptide (VII) (1 mg, 0.17 μmol was dissolved in 1.5 mL of Milli-Q water. Separately, 3 equivalent of Br-disialo-oligosaccharide (VIII, overnight dried) was dissolved (1.2 mg; 0.5 μmol) in 0.5 mL of Milli-Q water. Then peptide was added to this sugar solution in a dropwise manner (total volume of 2 mL). RP-HPLC and MALDI TOF MS analyses are shown in FIG. 3. The reaction was very slow but the major peak was found to be the desired product, glycoinsulin (structure IX of FIG. 2) with a minor product being the dimer of insulin (structure X, of FIG. 2). The peptide was purified after 24 hrs with one single injection into prep HPLC. Despite having a free thiol, thiol insulin does not scramble (1 μg/μl; tested between −4 to 25° C.). However, it has tendency to form dimer at high concentration and quantitive dimer formation was observed if thiol insulin (at 1 μg/μL water) is left at RT without Br-glycans for 24 h. The purity of glycoprotein was confirmed by analytical RP-HPLC (FIG. 3A) and the mass was confirmed by MALDI-TOF MS (m/z 8177.13, calcd. 8178.89, FIG. 3B). The yield of the glycosylation reaction was found to be 57.64% (˜60%) (0.8 mg of IX; 0.098 μmol.

### Example 3

### Quantification of Peptide Content

The actual peptide content of the glycoinsulin was determined using Direct Detect assay-free sample cards and the Direct Detect spectrometer (MerckMillipore). Each card contains hydrophilic spots surrounded by a hydrophobic ring to retain the analysed sample within the infrared beam for convenient sample application and analysis. All measurements were performed using 2 μL of sample solution. The glycoinsulin was found to have a 20% peptide content. The same concentration of glycoinsulin and native insulin was then injected into HPLC to confirm the same peak area under the curve and adjusted to 100% peptide content for both peptides. Then the adjusted net peptide content was used to prepare the samples for the following assays: insulin receptor binding assay, insulin tolerance test, atomic force microscopy fibrillation assay, circular dichroism spectroscopy and serum stability studies (discussed below).

### Example 4

### Insulin Receptor Binding Assay

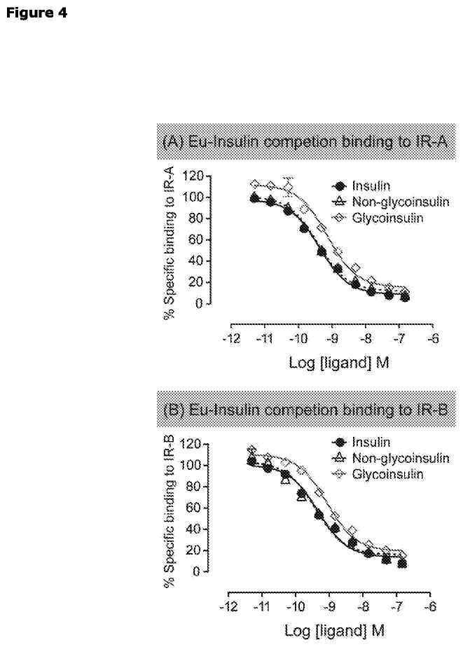

Receptor binding was measured as described in Denley et al., “Structural determinants for high-affinity binding of insulin-like growth factor II to insulin receptor (IR)-A, the exon 11 minus isoform of the IR”, Mol Endocrinol 2004, 18(10), 2502-12. Briefly, IGF-1R-negative cells overexpressing the insulin receptor B (IR-B) were generated. Cells were serum-starved for 4 h before lysis. Lysates were captured in a 96 well plate previously coated with anti-IR antibody. Approximately 500,000 fluorescent counts of europium-labelled insulin were added to each well along with increasing concentrations of unlabelled competitor and incubated for 16 h at 4° C. After washing time-resolved fluorescence was measured using 340 nm excitation and 612 nm emission filters with a BMG Lab technologies Polarstar fluorometer (Mornington, Australia). Binding curves are shown in FIG. 4; insulin and synthetic analogues curves are from four separate experiments with each point performed in triplicate. Results are expressed as a percentage of binding in the absence of competing ligand (% B/Bo), and the data points are the mean+/−SEM. Error bars are shown when greater than the size of the symbols. IC₅₀ values are shown in Table 1 below.

TABLE 1

| | IR-A | IR-B |
|---|---|---|
| | IC₅₀ (nM) | IC₅₀ (nM) |
| Insulin | 0.52 ± 0.09 | 0.43 ± 0.01 |
| Non-glycoinsulin | 0.60 ± 0.11 | 0.40 ± 0.06 |
| Glycoinsulin | 1.09 ± 0.34 | 0.89 ± 0.33 |

Competition binding assays as shown in FIG. 3 revealed that the binding affinities of the non-glycoinsulin (VI) for insulin receptor isoform A (FIG. 3A) and isoform B (FIG. 3B) (IR-A and IR-B) are equal to those of native insulin (Actrapid). The data confirm that modification at the N-terminal of the B-chain is tolerable. Glycoinsulin exhibits strong binding to insulin receptor (IR) isomers. There is a 2-fold poorer affinity of glycoinsulin (IX) for both receptors compared to insulin (FIG. 4) which suggests that the disialo modification interferes to a modest extent in the interaction with the IR.

Results are expressed as a percentage of binding in the absence of competing ligand (% B/Bo), and the data points are the mean+/−SEM. IC50 values are shown in Table 1. Non-glycoinsulin and glycoinsulin curves are from four separate experiments with each point performed in triplicate. Error bars are shown when greater than the size of the symbols.

### Example 5

### Insulin Tolerance Tests

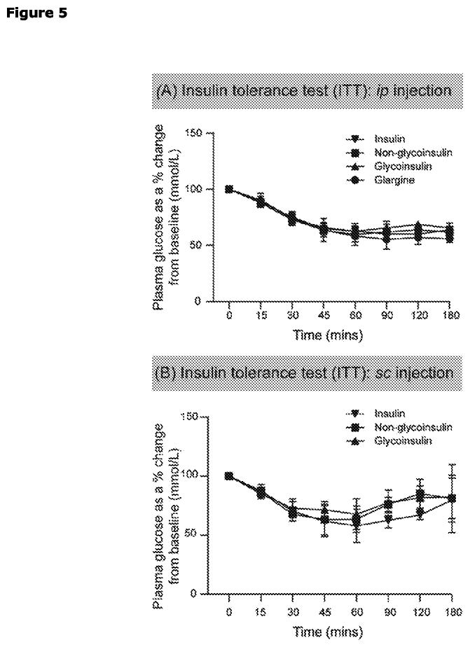

Insulin tolerance tests were performed as previously described in Won, N. et al., “Deficiency in interferon-gamma results in reduced body weight and better glucose tolerance in mice”, Endocrinology 2011, 152(10), 3690-9. Briefly, fed 8-10-week-old male C57BL/6J mice were anesthetised with sodium pentobarbitone (100 mg·kg−1) and injected intraperitoneally (i.p., FIG. 4A)) or subcutaneously (s.c., FIG. 4B) with 0.75 unit of insulin (actrapid), non-glycoinsulin, glycoinsulin or glargine per kg body weight. Blood samples were taken from the tail vein, and glucose concentration was measured using a glucometer (Precision Q.I.D., MediSense, MA, USA). The results are shown in FIG. 5. Results are presented as mean±SEM (n=4-5).

There was no significant difference between the peptides at any time point in terms of glucose lowering activity (data analysed by Two-way ANOVA, followed by Tukey's multiple comparison test (GraphPad Prism8.2.1)). The data clearly show that glycoinsulin is active in vivo and lowered blood glucose over a 3 h insulin tolerance test. Consistent with in vitro binding affinity results, the glucose-lowering efficacy of glycoinsulin seems to be slightly less, although not statistically significant, compared with native insulin (Actrapid). Intriguingly, the glucose-lowering effects of glycoinsulin are shown to be comparable to the long-acting insulin drug (synthetic glargine, as prepared in Hossain, M. et al., “Use of a temporary ‘solubilizing’ peptide tag for the Fmoc solid-phase synthesis of human insulin glargine via use of regioselective disulphide bond formation” Bioconjug Chem 2009, 20 (7), 1390-6) as demonstrated by i.p. injection data (FIG. 5A).

### Example 6

### Atomic Force Microscopy Studies for Fibrillation

For atomic force microscopy experiments, 200 μM and 50 μM insulin solutions were prepared by dissolving human insulin powder in a buffer solution (50 mM KCl/HCl in Milli-Q water, pH 1.6). Insulin solutions were incubated in polypropylene microcentrifuge tubes at 60° C. for a maximum of 24 h. After desired time intervals, 10 μL of insulin solution was transferred to a polypropylene microcentrifuge tube and diluted to different concentrations with 50 mM KCl/HCl buffer solution and quenched to 0° C. to rapidly inhibit further aggregation. Then, 5 μL diluted insulin solution was placed on a freshly cleaved mica substrate. Insulin aggregates were allowed to adsorb on mica for 5 mins. The excess insulin aggregates were washed dropwise with Milli-Q water for 2-3 times and then dried with gentle flow of nitrogen. Topographic images of insulin aggregates were collected with a Dimension iCon Atomic Force Microscopy (Bruker, Billerica, MA, USA). Tapping mode atomic force microscopy (AFM) images were recorded using silicon cantilevers (model RTESPA, Veeco, Santa Barbara, CA) with resonance frequency ˜300 kHz, nominal tip radius 8 nm and nominal spring constant 42 N/m.

Representative AFM height images are shown in FIG. 6, of (A) 50 μM insulin (Actrapid) solution and (B) 200 μM insulin solution incubated at 60° C. for 6 hours and 8 hours, and of (C) 200 μM glycoinsulin incubated at 60° C. for 6 hours and 8 hours. The vertical colour scale is 10 nm. Scan size: 3×3 μm². Each of the images of FIG. 6 are representative of 3-5 scans at different locations with varying scan sizes from 1-10 micron.

From FIG. 6 it can be seen that insulin forms fibrils at all time points tested at low and high concentrations (50 μM and 200 μM, FIG. 5A and FIG. 5B), but no fibrils were detected with glycoinsulin over the same time period (even at 200 μM, FIG. 5C). Furthermore, it is shown that glycoinsulin does not form fibrils even at 60° C. (FIG. 5C). As such, useful thermostable insulin analogues are provided, which have particular benefit for people living in poor tropical regions where reliable refrigeration is often not ubiquitous.

### Example 7

### Circular Dichroism Studies

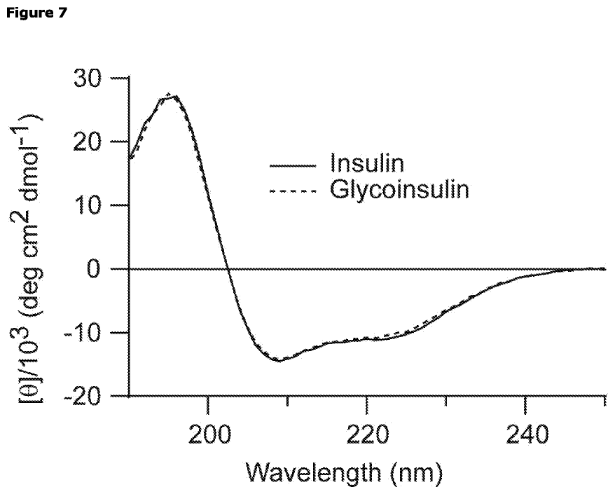

Modifications can cause conformational changes to the peptides that can expose sensitive amide linkages to proteases. The secondary structure of glycoinsulin was therefore analysed by circular dichroism (CD) spectrometry. CD spectra data of insulin and glycoinsulin were recorded using a Aviv Model 410 (Piscataway, NJ) spectrophotometer at 25° C. using 1 mm path length cell. The peptides were dissolved in 10 mM phosphate buffer at pH 7.5. The parameters used to obtain the spectra were wavelengths 190 to 250 nm with a data pitch of 0.1 nm, continuous scanning mode at a speed of 50 nm per minute and the number of accumulations taken per peptide was 3. The concentration of peptide used was 0.1 μg/μL for insulin and 0.17 μg/μL for glycoinsulin. This concentration was converted to molar concentration when calculating mean residue weight ellipticity for comparing the CD data and calculating helicity, as discussed below. The CD spectra are shown in FIG. 7.

The CD spectra showed a typical α-helical pattern with double minima at 208 and 222 nm (FIG. 6A). The mean residual ellipticity at 222 nm, [θ]₂₂₂, was used to calculate the helix content. The [θ]₂₂₂ values for glycoinsulin and human insulin were found to be −10564.677, and −11115.7 which correspond to an α-helix content of 30% and 31% respectively. Clearly, there was no observable perturbation in the secondary structure of glycoinsulin compared with native human insulin.

### Example 8

### Serum Stability Studies

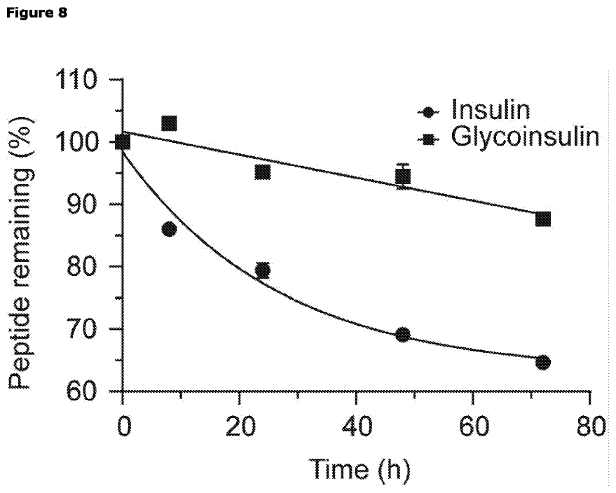

Finally, the in vitro serum stability of glycoinsulin was examined and compared with native human insulin for 72 h at 37° C. (FIG. 8). There were significant differences between the peptides at any time point (P value=0.0216, t-test). One-phase exponential decay and paired t-test analyses were performed with GraphPad Prism 8.0.2. The preliminary data show that disialo-glycosylation confers increased serum stability, which may be due to either or both of direct shielding from proteolytic enzymes by the glycan or by increased binding to serum albumin.

The structural and serum stability data fit well with high affinity IR binding in vitro and glucose lowering effects in vivo. The slight decrease in the binding affinity is likely due to steric effects from the bulky glycan that is close to the binding site.

As demonstrated by the above Examples, a homogeneous, mono-glycosylated insulin analogue that exhibits high activity both in vitro and in vivo has been designed and successfully synthesised. Importantly, it has been shown, for the first time, that disialo-glycoinsulin (IX of FIG. 1) does not form fibrils at both high concentration and high temperature. The disialo-glycoinsulin has a similar insulin-like fold and exhibits improved stability in human serum. This analogue is thus an attractive lead candidate for use, for example, in insulin pumps and more generally for the treatment of diabetes and for maintenance of blood glucose levels.

### Example 9

### Synthesis of Glucagon and Glycoglucagon

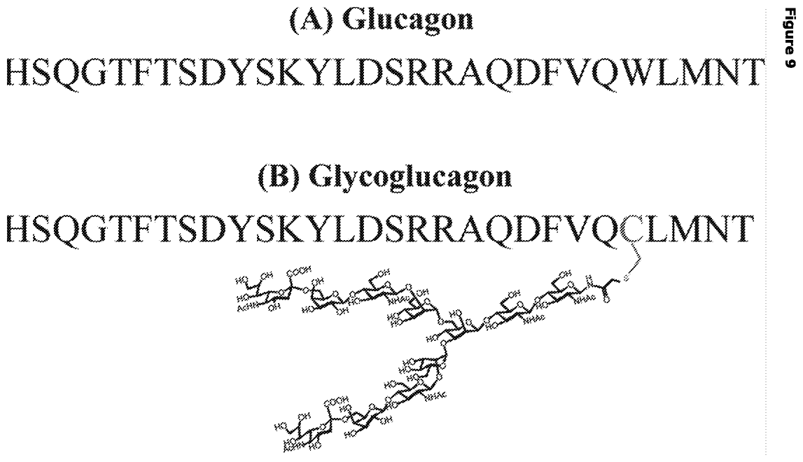

A glycoglucagon peptide was prepared using a method analogous to that described in Examples 1 and 2 for the preparation of glycoinsulin. The structures of the prepared glycoglucagon, as well as native glucagon, are shown in FIG. 9.

The linear backbone of human glucagon (SEQ ID NO:5) and its modified version (glucagon-W25C: Trp 25 was replaced with Cys 25 (SEQ ID NO:6)) were synthesised using a Biotage® Initiator+ Alstra microwave synthesiser (Biotage, Sweden), which adopts the standard Fmoc/tBu-based solid phase synthesis strategy. N^{α}-Fmoc deprotection was achieved by 20% piperidine in DMF at 40° C. for 2×5 min, and amino acid coupling (4 eq.) was achieved using HCTU (4 eq. in DMF) and DIEA (1 M in DMF) for 5 minutes. To reduce aggregation during synthesis, the Thr⁷ and Ser⁸ residues were coupled by manually treating with the dipeptide Fmoc-Thr(tBu)-Ser(Ψ^{Me, Me})-OH (2 eq.) in the presence of PyClock (2.5 eq.) and DIEA (1 M in DMF, 5 ml) for 4 hours. The completed peptidyl-resin was cleaved by treating with a cleavage mixture comprised of TFA/anisole/DODt/TIPS (94/3/2/1, 20 ml) for 3 hours. The crude peptide was then filtered, concentrated using stream of N₂, precipitated in cold ether, centrifuged and air dried.

The crude peptides were purified on a Phenomenex Gemini® C18 column (5 μm, 110 Å, 150×21.2 mm) using a Waters 600 semi-preparative RP-HPLC that incorporates a Waters 996 UV detector. Detection wavelength was set at 214 nm. Three different methods were used:

| | Buffer A | Buffer B | Gradient | Flow rate |
|---|---|---|---|---|
| 1 | 0.1% TFA | 0.1% TFA in ACN | 20-50% buffer B | 10 ml/min |
| | in water | | over 40 min | |
| 2 | 0.1% TFA | 0.1% TFA in ACN | 20-50% buffer B | 10 ml/min |
| | in water | | over 30 min | |
| 3 | 19 mM TEA | 60% ACN | 30-100% buffer B | 10 ml/min |
| | in water | in buffer A | over 40 min | |

Glucagon. Human glucagon peptide (SEQ ID NO:5) was synthesised on Fmoc-Thr(tBu)-TentaGel resin (0.18 mmol/g, 0.1 mmol scale). Purification was performed using method 1 followed by 3, and then lyophilised to obtain a white powder (7 mg). MW (calc.)=3482.75, MW (MALDI-TOF)=3484.2443.

Glycoglucagon. The linear backbone of glucagon-W25C (SEQ ID NO:6) (0.05 mmol scale) was synthesised using the same strategy as described for native human glucagon. After cleavage, the crude peptide was purified using method 1 to obtain a white powder (35.4 mg). The purified peptide (5 mg) and the bromo-glycan (3 eq., 10 mg) used in Example 2 was dissolved in triethylammonium acetate buffer (8 ml, pH=7.2), and stirred at 37° C. overnight. Purification using method 1 resulted in a white powder (2.4 mg). MW (calc.)=5663.66, MW (MALDI-TOF)=5664.8073.

### Example 10

### cAMP Activity Assays

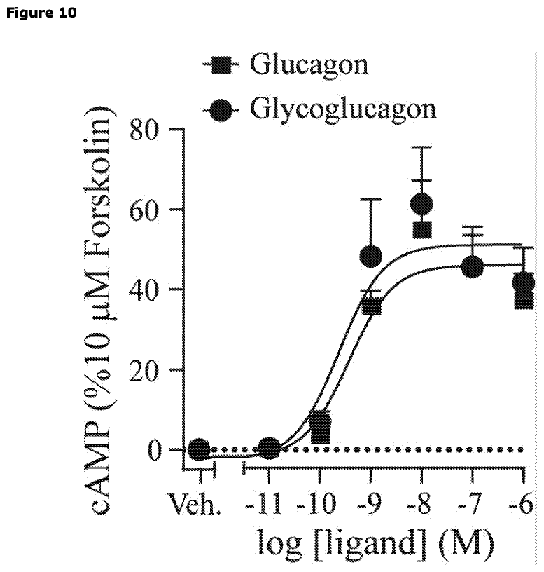

HEK293AWT cells were transiently transfected with pcDNA3-hGCGR, 48 hours post transfection, cells were challenged with increasing concentrations of each of glucagon and glycoglucagon prepared in Example 9 (−12-6M) for 30 minutes. cAMP level was evaluated using LANCE assay (Perkin Elmer). Results are shown in FIG. 10. The data is normalised to maximum response of FSK (10 uM) control.

As can be seen from FIG. 10, glycoglucagon. retains biological activity at the glucagon receptor relative to glucagon.

### Example 11

### Atomic Force Microscopy Studies for Fibrillation

For atomic force microscopy experiments, 287 mM solutions of glucagon and glycoglucagon prepared by dissolving each of glucagon and glycoglucagon obtained in Example 9 in Milli-Q water, adjusted to pH 2 with HCl were incubated at 37° C. for four days. Topographic images of glucagon aggregates were collected with a Dimension iCon Atomic Force Microscopy (Bruker, Billerica, MA, USA). Tapping mode atomic force microscopy (AFM) images were recorded using silicon cantilevers (model RTESPA, Veeco, Santa Barbara, CA) with resonance frequency ˜300 kHz, nominal tip radius 8 nm and nominal spring constant 42 N/m.

Representative AFM height images are shown in FIG. 11, of (A) glucagon (287 mM) and (B) glycoglucagon (287 mM) incubated at 37° C. for 4 days. The vertical colour scale is (A) 6 nm and (B) 3 nm. Scan size: 10×5 μm². Each of the images of FIG. 11 are representative of 3-5 scans at different locations with varying scan sizes from 1-10 micron.

From FIG. 11 it can be seen that native glucagon forms fibrils, but that fewer or no fibrils were detected in the chemically engineered glycoglucagon of Example 9.

1. A method for glycosylating a peptide or protein, comprising the steps of: a) providing a peptide or protein comprising an amino acid residue comprising an unprotected nucleophilic group; and b) reacting the nucleophilic group with a glycosyl moiety substituted with a leaving group to provide a glycosylated peptide or protein.

2-43. (canceled)


SEQUENCE LISTING (6 sequences)

SEQ ID NO: 1 - protein, 12 aa, HIV-1
GRKKRRQRRRPQ

SEQ ID NO: 2 - protein, 30 aa, Homo sapiens
FVNQHLCGSHLVEALYLVCGERGFFYTPKT

SEQ ID NO: 3 - protein, 31 aa, Artificial sequence
  Synthetic sequence
CFVNQHLCGSHLVEALYLVCGERGFFYTPKT

SEQ ID NO: 4 - protein, 21 aa, Homo sapiens
GIVEQCCTSICSLYQLENYCN

SEQ ID NO: 5 - protein, 29 aa, Homo sapiens
HSQGTFTSDYSKYLDSRRAQDFVQWLMNT

SEQ ID NO: 6 - protein, 29 aa, Artificial sequence
  Synthetic sequence
HSQGTFTSDYSKYLDSRRAQDFVQCLMNT